(19) United States
(12) Patent Application Publication
(10) Pub. No.: US 2003/0031326 A1
(43) Pub. Date: Feb. 13, 2003

(54) Magnet assembly for an electrolarynx

(76) Inventor: Richard Lukacovic, Portage, IN (US)

Correspondence Address: LOUIS WOO, LAW OFFICE OF LOUIS WOO, 717 NORTH FAYETTE STREET, ALEXANDRIA, VA, 22314, US

(21) Appl. No.: 09/923,832

(22) Filed: Aug. 7, 2001

(51) Int. Cl. A61F 2/20

(52) U.S. Cl. 381/70; 623/9

(57) ABSTRACT

A speech aid device having a housing and a solenoid disposed in the housing operatively coupled with a sounding head for movement therewith. A magnet assembly is mounted in the housing adjacent to the solenoid and operatively associated therewith. The magnet assembly has first and second pole pieces with a magnet disposed therebetween. A threaded fastener engages the first and second pole pieces and the magnet.

### BACKGROUND OF THE INVENTION

The present invention relates to a speech aid device for persons who have had a laryngectomy, and more particularly to portions of the sound generating mechanism of such a device.

Conventional speech aid devices are similar in size to that of a small flashlight. The device is cylindrical in shape and is battery operated so that the device is portable. The speech aid device has a sound generating mechanism which includes a circuit board, a solenoid assembly, a magnet assembly, a soft diaphragm, and a sounding head. The solenoid is a vibration producing coil associated with the sounding head to create vibrations in the sounding head. The sound generating mechanism produces a tone which, when the sounding head is applied to the user's throat, is conducted through the anterior aspect of the neck into the oral-pharyngeal cavity. The tone can be modulated by manipulation of the tongue and pharynx to produce intelligible speech.

Generally, the speech aid device includes an audio-frequency generator and amplifier which, together with potentiometers, allow for regulation of the pitch and loudness of the tone generated by the device. During use, the operator may alter the pitch and loudness of the speech as desired.

Several components of the conventional sound generating mechanism including the magnet assembly, soft diaphragm, and solenoid lack durability. These components are vulnerable to damage over time or if the device is subjected to an impact force such as when the device is dropped.

One problem with current speech aid devices is the construction of the magnet assembly. The magnet assembly includes a cup-shaped pole piece on which the magnet is seated. A second, upper pole piece is positioned on top of the magnet. The pole pieces and the magnet are glued together. A significant impact to the speech aid device such as when the device is dropped may cause the magnet to become dislodged and off-center from the cup-shaped pole piece. If this occurs, the performance of the speech aid device will diminish and complete failure of the device is possible.

An additional problem with current speech aid devices is the construction of the soft diaphragm. This soft membrane is a planar sheet of rubber latex which is secured to the inside of the device housing. During operation of the device, the diaphragm is repeatedly stretched through vibrational movement of the post of the solenoid which reduces the life of the diaphragm.

Another problem with the current speech aid device is the construction of the solenoid. The ends of the coil wire are connected by solder to lead wires which electrically join the solenoid and the circuit board. A top is placed adjacent the upper end of the coil to support the post of the solenoid. The wire comes off the coil and over the edge of the top to be electrically connected to the lead wire. The portion of the coil wire located over the edge of the top may be in contact with the inner surface of the solenoid chamber creating friction between the wire and the chamber as the solenoid vibrates. Further, if the speech aid device is dropped, the wire may contact the inside of the solenoid chamber and may break resulting in failure of the device.

An additional problem exists with the electrical connection between the coil wire and the lead wire. The connection of these wires is glued or melted to the upper surface of the solenoid top by any suitable method. Over time, the coil wire and lead wire connection may come loose from the solenoid top, leaving the electrical connection susceptible to damage during operation of the device. If this electrical connection is broken, the device is inoperable.

It is desired to provide a speech aid device with improved durability of the components of the sound generating mechanism.

### SUMMARY OF THE INVENTION

The present invention provides a speech aid device having a sound generating mechanism including a circuit board, a solenoid, a magnet assembly, a soft diaphragm, and a sounding head. When power is supplied to the solenoid, a magnetic field with the opposite polarity of the magnet assembly is generated causing the solenoid to move away from the magnet assembly. The magnet assembly includes a cup-shaped pole piece which supports a magnet thereon. A second upper pole piece is positioned atop the magnet. An aperture is formed through the pole pieces and the magnet to receive a threaded fastener, removably securing the pole pieces and the magnet. The movement of the solenoid produces vibrations which move a post secured to the solenoid into and out of contact with the sounding head to create vibrations thereof. With the speech aid device placed against the neck of the user, manipulations of the tongue and pharynx create intelligible speech.

The present invention provides a speech aid device having a housing with two ends. A solenoid is disposed in the housing and a sounding head operatively coupled with the solenoid for movement therewith is secured to one end of the housing. A magnet assembly is mounted in the housing adjacent the solenoid and operatively associated therewith. The magnet assembly includes first and second pole pieces with a magnet disposed therebetween. A threaded fastener engages the first and second pole pieces and the magnet.

The present invention also provides a speech aid device having a housing with two ends. A solenoid is disposed in the housing and a sounding head operatively coupled with the solenoid for movement therewith is secured to one end of the housing. A magnet assembly is mounted in the housing adjacent the solenoid and operatively associated therewith. The magnet assembly includes first and second pole pieces with a magnet disposed therebetween. The magnet has opposing surfaces each of which are in abutting relationship with one of the first and second pole pieces. An aperture is located in each of the first and second pole pieces and the magnet. The apertures align to receive a screw which threadedly secures the first and second pole pieces and the magnet.

The present invention further provides a magnet assembly for a speech aid device including a first pole piece having an aperture. A magnet having an aperture and opposing surfaces is positioned on the first pole piece with one of the opposing surfaces in abutting relationship with the pole piece such that the apertures align. A second pole piece having an aperture is positioned atop the magnet with one of the opposing surfaces in contact with the second pole piece such that the apertures align. A threaded fastener is located in the apertures removably securing the first and second pole pieces and the magnet.

One advantage of the present invention is that by using a threaded fastener to secure the components of the magnet assembly to one another, the chance of the magnet becoming dislodged or offset from the pole pieces is reduced.

An additional advantage of the present invention is that the pole pieces and magnet are removably secured to one another.

### BRIEF DESCRIPTION OF THE DRAWINGS

The above mentioned and other features and objects of this invention, and the manner of attaining them, will become more apparent and the invention itself will be better understood by reference to the following description of an embodiment of the invention taken in conjunction with the accompanying drawings, wherein:

FIG. 1 is a perspective view of the speech aid device in accordance with the present invention;

FIG. 2 is a sectional view of the speech aid device of FIG. 1;

FIG. 3 is a sectional view of a magnet assembly of the speech aid device of FIG. 1;

FIG. 4A is a sectional view of the soft membrane diaphragm of the speech aid device of FIG. 1;

FIG. 4B is a top plan view of the soft membrane diaphragm of FIG. 4A along line 4B-4B;

FIG. 5 is a side view of a solenoid of the speech aid device of FIG. 1; and

FIG. 6 is a top plan view of the solenoid of FIG. 5 along line 6-6.

Corresponding reference characters indicate corresponding parts throughout the several views. Although the drawings represent embodiments of the present invention, the drawings are not necessarily to scale and certain features may be exaggerated in order to better illustrate and explain the present invention.

### DESCRIPTION OF THE INVENTION

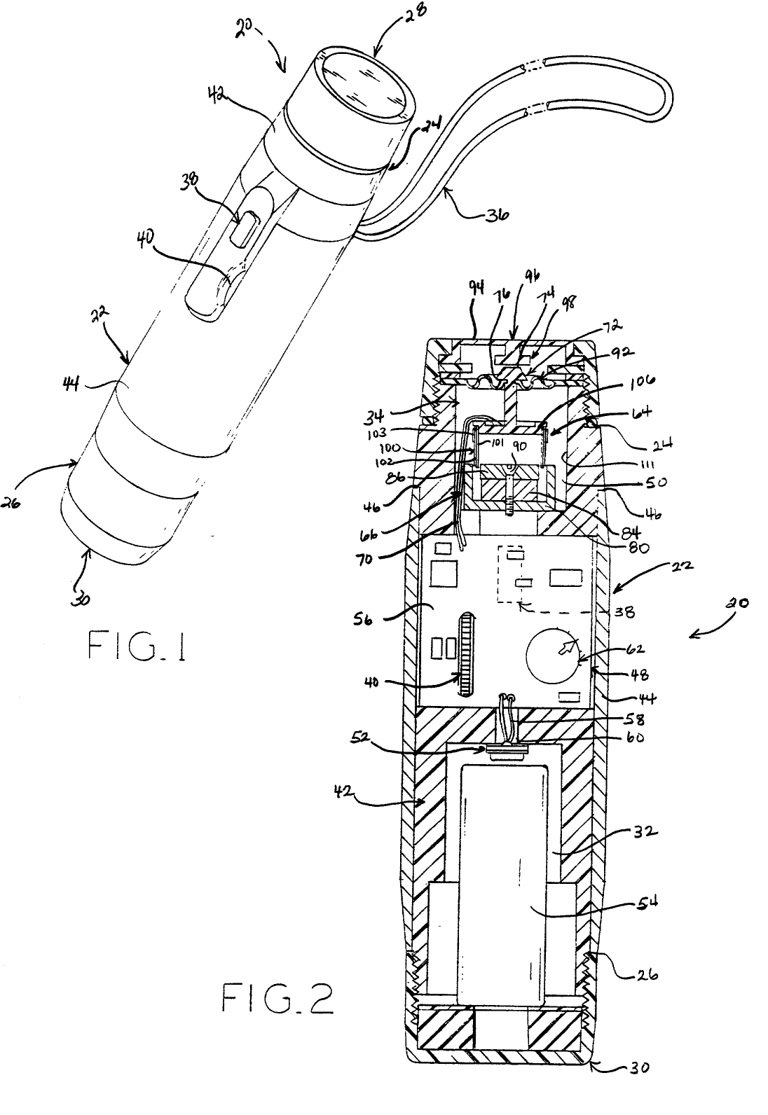

Referring to FIGS. 1 and 2, speech aid device or electrolarynx 20 includes housing 22 having ends 24 and 26 to which sounding head 28 and battery compartment cap 30 are respectively attached. Battery operated electrolarynx 20 has lanyard 36 secured to housing 22, 180° from activation button 38, allowing electrolarynx 20 to be portable and conveniently carried. Housing 22 includes activation button 38 to operate electrolarynx 20 and volume control dial 40 allowing the operator to control the loudness of the device.

Referring to FIG. 2, housing 22 includes inner portion 42 and sleeve 44. With battery compartment cap 30 removed, sleeve 44 may be slid over the length of inner housing portion 42 until contacting shoulder 46. Battery compartment cap 32 is then replaced to hold sleeve 44 in place. Sleeve 44 conceals the majority of inner housing portion 42, leaving activation button 38 and volume control dial 40 accessible. Sounding head 28 and battery compartment cap 30 are threaded onto ends 24 and 26 of inner housing portion 42. Sounding head 28 and battery compartment cap 30 are removably attached to inner housing portion 42 to allow for replacement of battery 54 and easy access to several components of sound generating mechanism 34.

Sound generating mechanism 34 includes circuit board 56, solenoid 64, magnet assembly 66, soft diaphragm 68, and sounding head 28. With the exception of sounding head 28, the components of sound generating mechanism 34 are located within circuit board compartment 48 and solenoid/magnet assembly compartment 50 defined in inner housing portion 42. Also located within inner housing portion 42 is battery compartment 32 having battery contacts 52 located at the end of compartment 32 nearest circuit board compartment 48. Battery 54 is positioned in compartment 32 and held firmly against contacts 52 by battery compartment cap 30.

Battery contacts 52 are electrically connected to circuit board 56 of sound generating mechanism 34 by wires 58 extending through passage 60 in inner housing portion 42. Circuit board 56 has mounted thereon activation button 38, volume control dial 40, pitch control dial 62, and a generator-amplifier (not shown). Activation button 38 is a push button switch which when depressed operates electrolarynx 20. Volume control dial 40 and pitch control dial 62 are connected to a pair of potentiometers (not shown) which regulate the pitch and loudness of the tone produced by electrolarynx 20. Activation button 38 and the pair of potentiometers provide inputs and variables to the generator-amplifier which supplies repetitive current pulses to solenoid 64 located in solenoid/magnet assembly compartment 50. Solenoid 64 is electrically connected to circuit board 56 via lead wires 70.

Solenoid 64 includes top 106 having post 72 secured in the center of upper surface 108 of top 106, standing approximately perpendicularly thereto. Top 106 and post 72 may be constructed from any suitable nonconductive material including plastic. Solenoid post 72 is integrally formed with top 106 having end 74 which contacts sounding head 28 and annular groove 76 which receives soft diaphragm 68. Soft diaphragm 68 is provided with hole 78 in the center thereof (FIGS. 4A and 4B). Hole 78 is stretched to have a diameter wide enough to receive post 72. Post 72 extends through hole 78 until diaphragm 68 is aligned with groove 76. Hole 78 is allowed to retract to its normal diameter and is captured in groove 76 as shown in FIG. 2. Diaphragm 68 is supported by end 24 of inner housing portion 42. When sounding head 28 is threaded onto end 24 of inner housing portion 42, washer 79 is positioned over diaphragm 68 and both washer 79 and diaphragm 68 are clamped between sounding head 28 and inner housing portion 42. Diaphragm 68 may also be secured to inner housing portion 42 using fasteners which would extend through apertures 81. Solenoid 64 is supported in its neutral position above magnet assembly 66 by diaphragm 68. Diaphragm 68 is constructed from any suitable elastomer including silicone, for example, which is stable at elevated operating temperatures and which does not degrade over time. Referring to FIGS. 2 and 4, diaphragm 68 is provided with annular ridges or undulations 92 which unfold through vibrational movement of post 72 during operation of solenoid 64. Annular ridges 92 reduce stretching of diaphragm 68 and may be formed in diaphragm 68 by any suitable method including injection molding. As solenoid 64 vibrates, post 72 and diaphragm 68 oscillate therewith. Diaphragm 68 has a spring-like effect acting to control the movement of solenoid 64, keeping solenoid 64 centered, and returning solenoid 64 to its neutral position upon shut down of electrolarynx 20.

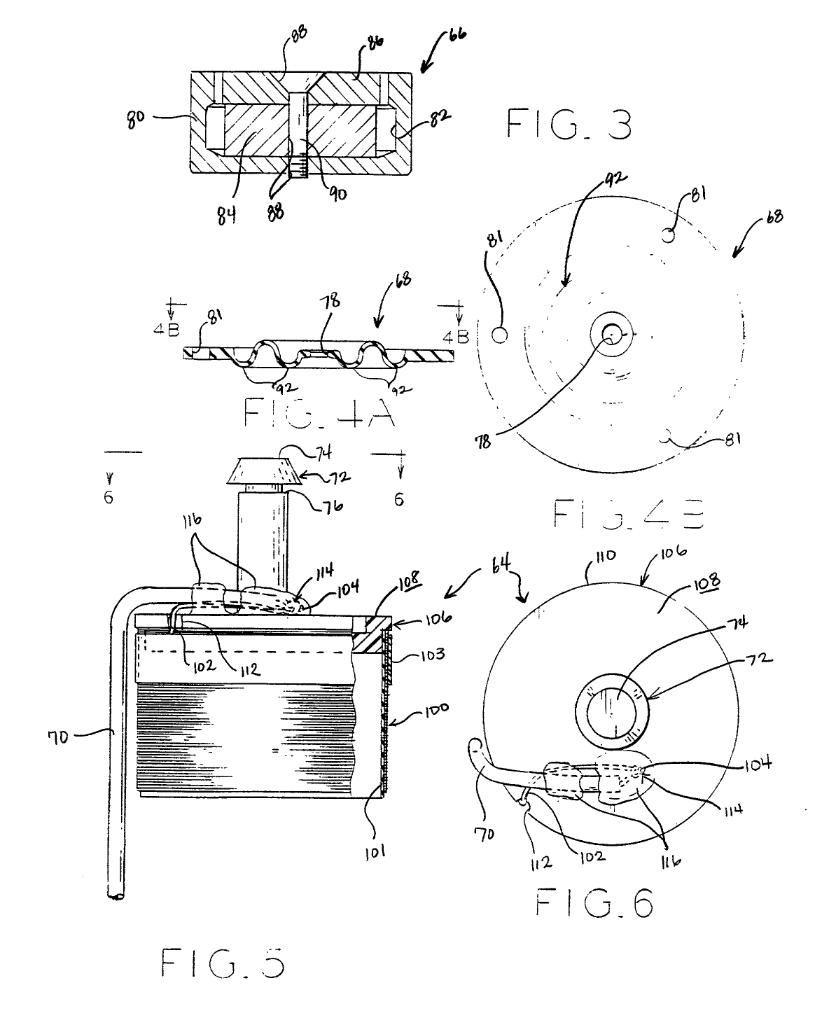

Also located in solenoid/magnet assembly compartment 50 is magnet assembly 66. Referring to FIGS. 2 and 3, supported by the bottom wall of solenoid compartment 50 is magnet assembly 66 which includes cup-shaped pole piece 80 having cavity 82 in which magnet 84 is received. Positioned atop magnet 84 is upper pole piece 86. Aperture 88 is provided through pole pieces 80 and 86, as well as magnet 84, to receive threaded fastener 90 which may be any suitable fastener including a screw or the like. By using a threaded fastener and adhesive, pole piece 80, magnet 84, and pole piece 86 are secured to one another.

Sounding head 28 of sound generating mechanism 34 includes hard diaphragm 94, abutment 96, and head 98. Abutment 96 extends downwardly from the center of hard diaphragm 94 with head 98 attached to the free end of abutment 96 approximately perpendicularly thereto. During operation of solenoid 64, end 74 of post 72 repeatedly contacts abutment 96 causing vibration of hard diaphragm 94. End 74 has a flat end which mates with the flat surface of head 98. Alternatively, end 74 may have a convex shape which mates with a concave shape of head 98. Further, a pad constructed from a resilient material, such as silicone, may be attached to end 74 and abutment 96 to help reduce extraneous noise created during contact thereof. Referring to FIGS. 5 and 6, solenoid 64 includes coil 100 wound about cylindrical support 101. A portion of coil 100 is conventionally covered with paper tape 103, for example. Coil 100 is constructed to have a cylindrical shape with coil wire 102 wound several times to create a plurality of adjacent layers of wire. End 104 of coil wire 102 comes off coil 100 to be attached to lead wires 70. Coil wire 102 must come over edge 110 of top 106 to secure to lead wire 70. In order to protect coil wire 102 from breakage due to contact with inner surface 111 of solenoid compartment 50 (FIG. 2), groove 112 is formed in top 106 by any suitable manner including being molded therein. Groove 112 is constructed to receive coil wire 102 to recess wire 102 inward from edge 110 of top 106.

End 104 of coil wire 102 is secured to lead wire 70 in any conventional manner including being soldered or twisted. In order to protect electrical connection 114 from fatigue due to the vibrations created during operation of electrolarynx 20, connection 114 is potted in silicone 116. Lead wires 70 are also partially potted in silicone 116 as the movement of solenoid 64 causes wires 70 to bend sharply several time a second which may weaken the wire. By potting lead wires 70 at an acute angle to top 106, the stress created in wires 70 during operation are more evenly distributed across the wire. Therefore, silicone 116 serves as a strain relief for wire 70 and dampens vibrations to preventing electrical connection 114 from becoming damaged or separated.

In operation, battery 54 supplies power to circuit board 56. Volume control dial 40 and pitch control dial 62 are both set to achieve the desired tone. When activation button 38 is depressed, repetitive current pulses are supplied to solenoid 64 by the generator-amplifier on circuit board 56 in accordance with the position of volume control dial 40 and pitch control dial 62. When power is supplied to solenoid coil 100, a magnetic field with the opposite polarity of magnet 84 is generated. This causes solenoid 64 to move away from magnet 84 so that end 74 of post 72 strikes abutment 96 of sounding head 28. The movement of abutment 96 creates movement of hard diaphragm 94. With activation button 38 depressed, solenoid 64 and thus hard diaphragm 94 continue to vibrate. With electrolarynx 20 placed against the neck of the user, manipulations of the tongue and pharynx create intelligible speech.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A speech aid device comprising:
a housing having two ends;
a solenoid disposed in said housing;
a sounding head secured to one end of said housing, said sounding head operatively coupled with said solenoid for movement therewith; and
a magnet assembly mounted in said housing, said magnet assembly located adjacent said solenoid and operatively associated therewith, said magnet assembly having first and second pole pieces, a magnet disposed between said pole pieces, and a threaded fastener engaging said first and second pole pieces and said magnet.

2. The device of claim 1, wherein said fastener is a screw.

3. The device of claim 1, wherein each of said first and second pole pieces and said magnet have an aperture therein, said apertures aligning to receive said fastener.

4. The device of claim 1, wherein said magnet further comprises a pair of opposing surfaces, each of said first and second pole pieces in abutting relationship with one of said opposing surfaces.

5. The device of claim 1, further comprising a circuit board operatively connected to said magnet assembly.

6. The device of claim 5, further comprising an electrical source electrically connected to said circuit board.

7. The device of claim 6, wherein said electrical source is a battery.

8. The device of claim 6, wherein said circuit board is coupled to an actuator, said solenoid being operative upon activation of said actuator.

9. A speech aid device comprising:
a housing having two ends;
a solenoid disposed in said housing;
a sounding head secured to one end of said housing, said sounding head operatively associated with said solenoid for movement therewith; and
a magnet assembly mounted in said housing, said magnet assembly located adjacent said solenoid and operatively associated therewith, said magnet assembly having first and second pole pieces, a magnet disposed between said pole pieces, said magnet having opposing surfaces, each of said first and second pole pieces in abutting relationship with one of said opposing surfaces, an aperture located in each of said first and second pole pieces and said magnet, said apertures aligning, a screw disposed in said apertures threadedly securing said first and second pole pieces and said magnet.

10. The device of claim 9, further comprising a circuit board operatively connected to said magnet assembly.

11. The device of claim 10, further comprising an electrical source electrically connected to said circuit board.

12. The device of claim 11, wherein said electrical source is a battery.

13. The device of claim 10, wherein said circuit board is coupled to an actuator, said solenoid being operative upon activation of said actuator.

15. A magnet assembly for a speech aid device comprising:
a first pole piece having an aperture;
a magnet having an aperture and opposing surfaces, said magnet positioned on said first pole piece with one of said opposing surfaces in abutting relationship with said first pole piece, said first pole piece aperture aligning with said magnet aperture;
a second pole piece having an aperture, said second pole piece positioned on said magnet, one of said opposing surfaces in contact with said second pole piece, said second pole piece aperture aligning with said magnet aperture; and
a threaded fastener located in said apertures removably engaging said first and second pole pieces and said magnet.

16. The magnet assembly of claim 15, wherein said fastener is a screw.
